(19) United States
(12) Patent Application Publication
(10) Pub. No.: US 2012/0016050 A1
(43) Pub. Date: Jan. 19, 2012

(54) Monoisocyanate-Acrylate Monomers and Products Ulitilizing the Same

(76) Inventors: Joseph A. Leon, West Simsbury, CT (US); Ahmet Nebioglu, Winsted, CT (US); James H. Aerykssen, Torrington, CT (US); Richard David Zopf, Torrington, CT (US); Igor V. Khudyakov, Hickory, CT (US)

(21) Appl. No.: 13/138,020

(22) PCT Filed: Dec. 24, 2009
(86) PCT No.: PCT/US2009/006709

§ 371 Date: Sep. 22, 2011

Related U.S. Application Data
(60) Provisional application No. 61/203,667, filed on Dec. 26, 2008.

(51) Int. Cl. C08F 220/34; C07C 269/02; C08F 2/46; C07C 271/22

(52) U.S. Cl. 522/96; 560/158; 526/301

(57) ABSTRACT

Urethane acrylate oligomers, suitable for use in coatings and like formulations, prepared by capping polyols having hydroxyl functionality (fOH) equal to or greater than 4, using 2-isocyanatoethyl acrylate or 2-isocyanatoethyl methacrylate, thereby avoiding the gelation that normally occurs in attempting to prepare urethane acrylates with high/OH polyols by reaction with diisocyanates. Reaction of low molecular weight polyols, containing two or three hydroxyl groups, with mono isocyanate(meth)acrylate monomers produces useful, low viscosity urethane (meth)acrylate oligomers. Specifically, capping of a mole of 2,2-dihydroxymethyl butanoic acid by two moles of 2-isocyanatoethyl acrylate molecules leads to the formation of radiation curable water-soluble liquid monomers that are transparent and soluble in water. Oligomers obtained by capping with 2-isocyanatoethyl acrylate demonstrate enhanced adhesion to glass and stainless steel. Solid, hydroxyl-containing chemicals can be transformed to liquids by reaction with monoisocyanate-(meth)acrylate monomers

### BACKGROUND OF THE INVENTION

Urethane acrylate oligomers are widely used as ingredients in radiation-curable formulations for producing films, coatings, adhesives, and the like. Products containing such oligomers may be highly flexible, elastomeric, and tacky; the oligomers may also serve as base resins, adhesion promoters, and reactive tackifiers in pressure-sensitive and laminating formulations that exhibit significantly improved adhesion to a wide variety of films and foils. Oligomers having such attributes are commercially available from Bomar Specialties Co., of Torrington, Conn.

Urethane acrylate oligomers (hereinafter sometimes referred to as “UAOs”) are commonly synthesized by reaction of a diisocyanate with a polyol having hydroxyl functionality (f_{oH}) of 2 to 3 at an equivalent ratio of approximately two isocyanate groups to one hydroxyl group, thus forming a urethane prepolymer. To provide radiation-curable oligomers, the urethane prepolymers are capped with an acrylate or methacrylate compound (i.e., a “(meth)acrylate” compound).

While high urethane functionality in such oligomers should make them valuable as ingredients for producing hard and abrasion-resistant coatings, it is most difficult, if not virtually impossible, to prepare urethane prepolymers from polyols with functionality of 4 or greater, not to mention dendrimers and dendritic polyols (which may have functionalities as high as 16), by a reaction with diisocyanates in ratios of —NCO and —OH equivalents of 2-3:1. Endeavoring to effect such reactions almost inevitably leads to gelation of the prepolymer, due to the high probability of that exists for chain extension and branching in a system with high f_{OH}*f_{NCO} and comparable numbers of equivalents of —NCO and —OH. It is known that the probability of gelation is directly proportional to the product of functionalities of monomers (oligomers), and inversely proportional to a ratio of equivalents r (see Hiemenz, P. C.; Lodge, T. P. Polymer Chemistry, CRC Press, Boca Raton, 2007):

p˜f_{OH}*f_{NCO}/r

Where r≧1.0, and r is the ratio of NCO equivalents to OH equivalents, if NCO>OH, or is the ratio of OH equivalents to NCO equivalents if OH>NCO.

A possible way to synthesize multifunctional UAOs with high f_{OH} polyols, however, is to cap the polyol with a monoisocyanate-(meth)acrylate. Such an agent, in which f_{NCO}=1, precludes gelation due to chain extension.

In addition to the likelihood of gelation discussed above, the methods commonly employed for synthesis of UOAs are not optimal and/or do not produce optimal properties in the products. Moreover, it would be desirable to extend the range of applications for UAOs beyond those that presently exist.

Monoisocyanate-(meth)acrylate monomers, such as 2-isocyanatoethyl acrylate and 2-isocyanatoethyl methacrylate (hereinafter sometimes referred to as IA and IMA), are known in the art and are available from Showa Denko K.K. under the designations “AOI-VM” and “Karenz-MOI,” respectively. U.S. Pat. Nos. 5,030,696 and 5,334,681 may be of interest in connection with the use of such monomers. It is also known that IM and IMA monomers can be employed for the “one-step” synthesis of urethane acrylate oligomers by reaction with suitable polyols.

### SUMMARY OF THE INVENTION

Broad objects of the present invention are to provide improved methods for synthesizing and utilizing monoisocyanate-(meth)acrylates; to enable expanded applications for such monomers; to provide novel isocyanate-based oligomers that lead to desirable properties in cured products in which they are employed; and to provide novel formulations and products containing such oligomers.

A more specific object of the invention is to provide isocyanate-based oligomers that are well suited for use in formulations that are curable to films, coatings, adhesives, and like solid products. The new oligomers may have reduced viscosity in comparison to similar isocyanate-based oligomers; they may afford significantly enhanced adhesion to certain substrates; and they may impart other desirable properties to products produced from formulations in which they are incorporated.

It has now been found that certain of the foregoing and related objects of the invention are attained by the provision of a method for the synthesis of UV-curable urethane (meth)acrylate oligomers, in one-stage, by capping of polyols with 2-isocyanatoethyl (meth)acrylates. Diols and higher polyols capped by 2-isocyanatoethyl (meth)acrylate monomers produce UAOs of much lower viscosity than similarly structured conventional urethane acrylate oligomers, and it is found that, in many instances, there is no need to employ a reactive diluent to produce coatings based on polyols capped by those monomers.

The invention also enables capping of multifunctional polyols (f_{OH}≧4), which usually gel during standard syntheses of urethane acrylate oligomers with diisocyanates, and different degrees of (meth)acrylation of OH-groups of the same multifunctional polyol (9-99%) allow selective synthesis of urethane acrylate oligomers having a wide range of properties.

The invention further enables the capping, with monoisocynate-(meth)acrylate monomers, of hydroxyl-functional monomers to obtain liquid urethane products that would normally produce solid products with conventional isocyanate methods. This invention enables obtaining liquid urethane (meth)acrylate functional monomers from commercially available photoinitiators having hydroxyalkyl substituent(s). The resultant functionalized photoinitiators copolymerize with (meth)acrylates, making any residual photoinitiators present non-leachable from the cured products produced. Another benefit of ability to obtain liquid urethane (meth)acrylate monomers is in synthesis of acid functional urethane (meth)acrylate monomers that are highly effective as adhesion promoters.

The urethane acrylate oligomers provided by the invention are highly beneficial for use in formulating very desirable UV-curable products. For example, multifunctional polyols (f_{OH}≧4) capped by 2-isocyanatoethyl (meth)acrylate monomers exhibit extremely high tensile moduli, and they demonstrate good adhesion to glass and stainless steel. Conventional isocyanate routes result in gelation, and therefore unusable products.

The reaction effected, in accordance with the present invention, of for example 2,2-dihydroxymethyl butanoic acid with 2-isocyanatoethyl acrylate (at a 1:2 molar ratio) is found to produce a monomer that is soluble in water in any concentration. Stable, transparent solutions are produced from the monomer in the presence of tertiary amines, and the polymerization of the monomer itself provides a product that exhibits good mechanical properties.

More particularly, in one embodiment of the invention a method for the production of useful urethane (meth)acrylate oligomers, without substantial gelation, comprises the steps: forming a reaction mixture comprised of a monoisocyanate-(meth)acrylate monomer and a polyol having an hydroxyl functionality of at least 4, the amount of the polyol being not significantly in excess of the amount of the monoisocyanate-(meth)acrylate monomer, on a stoichiometric basis; and effecting reaction between the monoisocyanate-(meth)acrylate monomer and the polyol to produce a urethane (meth)acrylate oligomer that is substantially free of gelation and in which, on an equivalent basis, at least about 70 percent of the hydroxyl groups of the polyol are capped with the monoisocyanate-(meth)acrylate monomer.

Normally, in carrying out the foregoing method the amount of the polyol will not exceed the amount of the monoisocyanate-(meth)acrylate monomer by more than about 30 percent, on an hydroxyl equivalent basis, and preferably in the amounts of the polyol and monoisocyanate-(meth)acrylate monomer will be substantially stoichiometrically equivalent. The method is especially beneficial in instances in which the polyol is a dendrimer, the (meth)acrylate oligomer produced being a hyperbranched (meth)acrylate oligomer. The monoiscocyanate-(meth)acrylate monomer employed in all embodiments of the invention will usually be selected from the group consisting of 2-isocyanatoethyl acrylate and 2-isocyanatoethyl methacrylate.

In another embodiment, the invention provides a method for the production of a liquid derivative from a solid starting chemical, comprising the steps: forming a reaction mixture comprised of an monoisocyanate-(meth)acrylate monomer and a solid starting chemical that contains hydroxyl functionality; and effecting reaction between the monoisocyanate-(meth)acrylate monomer and the solid starting chemical to produce a liquid derivative, the physical states of the derivative and the starting chemical being determined at room temperature.

The solid starting chemical utilized in the method will desirably be a low molecular weight diol. Especially desirable products are produced when the solid starting chemical is selected from the group consisting of 2,2-dihydroxymethyl butanoic acid, dimethylol acetic acid, dimethylol propionic acid, dimethylol pentanoic acid, and dimethylol hexanoic acid. Effecting reaction between 2,2-dihydroxymethyl butanoic acid and 2-isocyanatoethyl acrylate, at an acid: monomer molar ratio of 1.5:1.0, produces an especially useful product, which product may be further reacted with triethylamine to produce a water-soluble quaternary ammonium salt having uniquely desirable properties. The method is also effected to significant benefit when the solid starting chemical is a photoinitiator containing hydroxyalkyl groups.

A further embodiment of the invention provides a method for the production of useful, relatively low viscosity (meth)acrylate oligomers, comprising the steps: forming a reaction mixture comprised about 30 to 75 percent of a monoisocyanate(meth)acrylate monomer and about 25 to 70 percent of a polyol containing two or three hydroxyl groups, or a mixture of such polyols, the polyol having a molecular weight in the range 250 to 650 g/mol; and effecting reaction between the monoisocyanate(meth)acrylate monomer and the polyol to produce a urethane (meth)acrylate oligomer having a viscosity not higher than about 150 MPa. The polyol will desirably be monomeric and, again, the monoiscocyanate-(meth)acrylate employed will usually be either 2-isocyanatoethyl acrylate or 2-isocyanatoethyl methacrylate.

Other objects of the invention are attained by the provision of products produced by the foregoing methods, and still other objects are attained by the provision of solid polymeric products comprising the urethane (meth)acrylate oligomer so produced and a polymerizable diluent reactive with the oligomer. In the latter instances, the polymerizable diluent will usually comprise a (meth)acrylate monomer. Typically, the formulation will contain about 70 to 50 weight percent of the urethane (meth)acrylate oligomer and, conversely, about 30 to 50 weight percent of the reactive diluent. The formulation may desirably additionally include a catalyst for inducing free radical polymerization, which may be either a photoinitiator or a thermal initiator.

Further objects of the invention are attained by the provision of a water-reducible urethane acrylate monomer having the chemical structure.

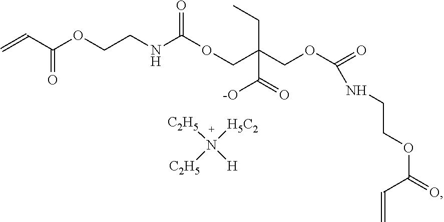

and additional objects are obtained by the provision of a mixture of two urethane acrylate monomers having the chemical structures.

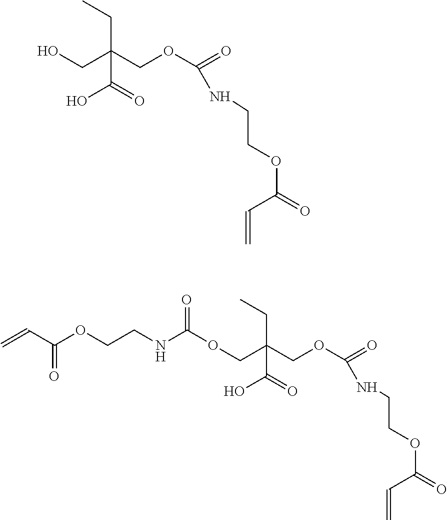

In the latter instance, the monomers will generally be present in a substantially equimolar (i.e., approximately 1:1) ratio.

### DETAILED DESCRIPTION OF THE PREFERRED EMBODIMENTS

Commercially available products employed in the examples described below, and the sources from which they are obtained, are:

As polyols: Voranol 220-028, of Dow; PolyTHF 250, of BASF; Teraphane 2000 and Terathane 2900, of Invista; Potymeg 30-168, of Arch; dendritic polyol, of Perstorp; and Boltorn P1000 (f_{oOH}=14), Boltorn P500 (f_{OH}=16), Boltorn H2004 (f_{OH}=6), and CAPA 4101 (f_{OH}=4). The 2-hydroxyethyl acrylate (HEA) utilized was of Osaka Organic Chemical; 2-hydroxyethyl methacrylate (HEMA) was of Evonik-Degussa; 2,2-dihydroxymethyl butanoic acid (dimethylol butanoic acid, or DMBA), and triethylamine (TEA), were of Aldrich. The 2-isocyanatoethyl acylate (IA) and 2-isocyanatoethyl methacylate (IMA) were of Showa Denko K.K. The aliphatic diisocyanates are known in the industry as H₁₂MDI (also known as Desmodur W or DesW) and IPDI, both of Evonik-Degussa; DI:TDI (80:20) was of Bayer; and a monoisocyanate-styrene derivative, 1-(1-isocyanto-1-methylethyl)-3-(1-methylethenyl)benzene (TMI), having the structural formula:

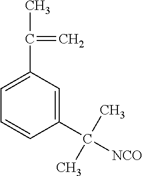

### was of Cytec.

As catalysts for urethane syntheses, either a dibutyltin dilaurate (DBTDL) product (Fascat 4202) or a stannous octoate product (Facsat 2003), both of Arkema, was used (with evidently indistinguishable results) at a concentration of about 500 ppm. The formulations were stabilized by addition of about 500 ppm MEHQ, of Eastman Chemical, to prevent spontaneous polymerization of the (meth)acrylate group-containing monomers and oligomers.

Reactive diluents employed were isobornyl acrylate (IBOA) and tripropyleneglycol diacrylate (TRPGDA), both of Sartomer. The products of Ciba Additives, designated Irgacure 184, Irgacure 819 DW (the latter, being water-soluble as a dispersion, being used only in aqueous solution), and Darocur 1173, were employed (as received) as UV-curing photoinitiators.

Although HEA, HEMA, TMI, IA, IMA are used as capping agents in the examples that follow, it should be appreciated that other compounds containing vinyl-isocyanate functionality can often be substituted (albeit with significantly disparate results in certain instances, as will be clear from the following description). Similarly, while IPDI, DesW, and TDI are utilized in the examples presented, it is expected that other diisocyanates, such as the MDI, TMXDI, TDI-100, HDI, and TMDI can be substituted, with comparable results.

Curing of coatings was effected in air, using a Fusion 300 W/in UV-processor fitted with a D-bulb. Three passes, at 20 ft/min, were usually employed, producing a total radiant exposure of the samples to UV-light of about 1 J/cm², measured using a PowerPuck radiometer. Cured (dry) films of thicknesses of either 25 or 200 μm were produced, for measurements of MEK double rubs and mechanical properties, respectively.

Properties of products were analyzed using gel permeation chromatography (GPC), which gives molecular weights as weight average and number average (M_{w} and Mₙ), and also the molecular weight distribution (MWD=M_{w}/Mₙ), and all compounds on a GPC trace were included in calculations of molecular weights; the GPC device and GPC experiments are described by Swiderski and Khudyakov (see Swiderski, K. W.; Khudyakov, I. V. Ind. Eng. Chem. Res. 2004, 43, 6281). A Perkin-Elmer Spectrum One model IR spectrometer, with a diamond crystal UATR, was used for obtaining spectral data. Viscosity (η) was measured using a Brookfield RVT unit with a small adapter (spindle SC4-15 and cup 7R) connected to a Neslab circulating water bath, at temperatures of 25 and 50° C. Tensile properties of cured samples (elongation to break, tensile strength at break, and tensile modulus) were measured with using a Cheminstruments Tensile Tester-1000, controlled by the Cheminstruments EZ-LAB system program, with the test method being designed so as to comply with ASTM D 822. At least five samples of each cured product were studied, at ambient temperature, to verify the reproducibility of data obtained.

Hardness values of the cured films were measured using a Type A or Type D durometer (PTC Instruments). All measurements were performed, again at room temperature, and all numerical values presented (except viscosity), were measured at room temperature as well.

Oligomer color was measured using the DR/2000 spectrophotometer of Hach, and the data obtained are presented in APHA units.

A conventional “pick test,” known in the industry, was employed to evaluate adhesion of cured oligomers to common substrates, and the number of “MEK double rubs” that the cured film could withstand was estimated. In the standard MEK double rubs test, one counts the number of double rubs that could be made, using a cloth wet with MEK and placed under a 16-ounce round ball hammer, before the moment when a film of the sample delaminates or is breached; the test is considered to be of modest accuracy.

Syntheses were typically carried out in a one-liter flask, and included one or two reactions of the carbamate (urethane) link formed between —NCO and —OH groups. The reactions, usually catalyzed by DBTDL or stannous octoate, were run at 60° C., and reactants were added in such manner as to maintain the reaction temperature below 70° C. The first stage of the two-stage reaction described occurred over a period of 2 hours, and the second, final stage occurred over a period of 8 hours.

Syntheses started with diisocyanate, the selected catalyst, and MEHQ in the pot, with the remaining ingredients being added subsequently. In a standard syntheses, urethane acrylates were prepared by a reaction of a polyol (hereinafter sometimes being designated “P”) with a diisocyanate (hereinafter sometimes being abbreviated “DI”), in a first stage, with subsequent capping by HEA or HEMA being effected in a second stage, the ratio of reagents employed P:D:HEA (or HEMA) being 1:2:1, on equivalent bases; f_{OH} and OH numbers provided by the suppliers were relied upon. The standard synthesis is described in more detail below, and is referred to as “direct addition” (see for example Swiderski, K. W.; Khudyakov, I. V., supra).

In a “reverse addition” synthesis, the diisocyanate is first reacted with a capping agent, followed by the introduction and reaction with the polyol ingredient (see again Swiderski, K. W.; Khudyakov, I. V., supra). Additional comments upon the syntheses employed are provided below, as appropriate.

Spectra of the reactive mixtures were obtained, paying particular attention to the peak at 2230 cm⁻¹ (−NCO), so as to determine the completeness of reaction, via extinction of isocyanate. Syntheses were deemed complete when the measurements showed less than 0.2% of the initial absorption of residual —NCO. The final products had a mass of 700-750 g, and each synthesis was repeated two or three times, using the same reactants, to verify the reproducibility of data.

The one-stage reaction of polyols, of given hydroxyl functionalities, with IA or IMA is even more straight-forward than is the synthesis of standard UAOs. More particularly, and by way of example, stoichiometric amounts of the selected polyol and monoisocyanate were charged to a reaction vessel so as to provide a total mass of 700-750 g. The reaction mixture was heated to 40 to 60° C., with stirring, and about 200 ppm of MEHQ (or a comparable amount of another common polymeration inhibitor, such as Irganox 1010, of Ciba Additives, and BHT, of Penta Manufacturing) was added to accommodate the high reactivity of IA and IMA towards OH-groups; a small amount (20 ppm is preferred) of DBTDL (or stannous octoate) was also added to the reaction mixture. The reaction usually comes to completion in about one to two hours, the state of which can easily be verified by IR monitoring.

As an alternative one-stage method, the adduct can be produced by a reaction carried out in the absence of catalyst. That can be done by heating the mixture at a temperature of about 60 to 65° C.; a period of about 8 hours is generally required.

1. Products of Reactions of High Functionality Polyols with IMA

Table 1 below presents properties of the multifunctional polyols used in the present examples:

TABLE 1
Functionalities and molecular weights of high functionality polyols*

| | Boltorn P1000 | Boltorn P500 | Boltorn H2004 | CAPA 4101 |
|---|---|---|---|---|
| f_{OH} | 14 | 16 | 6 | 4 |
| M_{W} | 1313 | 1048 | 3960 | 1613 |
| Mₙ | 458 | 363 | 2017 | 1284 |
| MWD | 2.87 | 2.89 | 1.96 | 1.26 |

*Determination error of M_{W}, Mₙ, and of MWD is 15%.

The following oligomers were produced: Boltorn P1000 capped with IMA, wherein 80% of the OH-groups, on an equivalent basis, were capped (designated Oligomer 1-1); Boltorn P500 capped with IMA, wherein 70% of the OH-groups, on an equivalent basis, were capped (designated Oligomer 1-2); Boltorn H2004 capped with IMA, wherein 95% of the OH-groups, on an equivalent basis, were capped (designated Oligomer 1-3), and CAPA 4101 capped with IMA, wherein 100% of the OH-groups, on an equivalent basis, were capped (designated Oligomer 1-4) Properties of the oligomers are summarized in the Table 2:

TABLE 2
Properties of oligomers prepared with high functionality polyols and IMA*

| | Oligomer | | | Oligomer |
|---|---|---|---|---|
| | 1-1 | Oligomer 1-2 | Oligomer 1-3 | 1-4 |
| M_{W} | 2374 | 2213 | 4378 | 2267 |
| Mₙ | 880 | 875 | 2270 | 2020 |
| MWD | 2.70 | 2.61 | 1.93 | 1.12 |
| Viscosity η @ | 310 | 1060 | 360 | 140 |
| 25° C., P | | | | |
| Color (APHA) | 10 | 0 | Light yellow, | 0 |
| | | | slight haze | |

*Determination error of M_{W}, Mₙ, MWD and of η is 15%.

The oligomers identified in Table 2 were diluted with IBOA, and cured in the manner described above. Properties of cured materials are presented in Table 3:

TABLE 3
Properties of cured formulations prepared
with high functionality polyols and IMA*

| | | Elonga- | Tensile | | | |
|---|---|---|---|---|---|---|
| | Tensile | tion to | Modu- | | MEK | η** @ |
| | Strength, | break, | lus, | Hard- | double | 25° C., |
| | MPa | % | MPa | ness | rubs | cP |
| Oligomer 1-1, | 46 | 13 | 1,190 | 85 D | 200 | 1,325 |
| 30% IBOA | | | | | | |
| Oligomer 1-2, | 58 | 5 | 1,560 | 87 D | 200 | 2,650 |
| 30% IBOA | | | | | | |
| Oligomer 1-3, | 15 | 39 | 113 | 66 D | 8 | 1,275 |
| 30% IBOA | | | | | | |
| Oligomer 1-4, | 37 | 10 | 901 | 82 D | 200 | 1,200 |
| 30% IBOA | | | | | | |

*Determination error of M_{W}, Mₙ and of MWD and η is 15%.
**η represents the viscosity of the formulation, with diluent.

As can be seen from the data set forth in the first four lines in Table 3, one-stage capping of the high functionality polyols with IMA leads to remarkably strong coatings.
2. Products of Reactions of High Functionality Polyols with IA

The following oligomers were produced: Boltorn P1000 capped with IA, wherein 80% of the OH-groups, on an equivalent basis, were capped (designated Oligomer 2-1); Boltorn P500 capped with IA, wherein 80% of the OH-groups, on an equivalent basis, were capped (designated Oligomer 2-2); Boltorn H2004 capped with IA, wherein 95% of the OH-groups on an equivalent basis, were capped (designated Oligomer 2-3); and Boltorn P1000 capped with IA, wherein 30% of the OH-groups on an equivalent basis were capped (designated Oligomer 2-4). Properties of the oligomers are summarized in the Table 4:

TABLE 4
Properties of oligomers prepared with Boltorn polyols and IA*

| | Oligomer | | | Oligomer |
|---|---|---|---|---|
| | 2-1 | Oligomer 2-2 | Oligomer 2-3 | 2-4 |
| M_{w}, g/mol | 2,350 | 2,050 | 4,370 | 1650 |
| Mₙ, g/mol | 815 | 810 | 2543 | 610 |
| MWD | 2.67 | 2.54 | 1.72 | 2.70 |
| Viscosity η @ | 280 | 1,060 | 320 | 58 |
| 25° C., P | | | | |
| Color | 0 | 10 | Light yellow, | 0 |
| (APHA) | | | slight haze | |

*Determination error of M_{W}, Mₙ and of MWD and η is 15%.

The oligomers identified in Table 4 were diluted with IBOA or TRPGDA, and cured as described above. Properties of the cured materials are presented in Table 5:

TABLE 5
Properties of cured formulations prepared
with Boltorn polyols and IA*

| | | Elonga- | Tensile | | | |
|---|---|---|---|---|---|---|
| | Tensile | tion to | Modu- | | MEK | η @ |
| | Strength, | break, | lus, | Hard- | double | 25° C., |
| | MPa | % | MPa | ness | rubs | P** |
| Oligomer 2-1, | 36 | 7 | 978 | 85 D | 200 | 14.75 |
| 30% IBOA | | | | | | |
| Oligomer 2-1, | 27 | 6 | 734 | 83 D | 200 | 3 |
| 50% TRPGDA | | | | | | |
| Oligomer 2-2, | 37 | 4 | 1134 | 82 D | 200 | 4 |
| 50% TRPGDA | | | | | | |
| Oligomer 2-3, | 9 | 47 | 34 | 59 D | 36 | 12.75 |
| 30% IBOA | | | | | | |
| Oligomer 2-3, | 15 | 17 | 157 | 70 D | 27 | 2.25 |
| 50% TRPGDA | | | | | | |
| Oligomer 2-4, | .95 | 14 | 9 | 69 A | 15 | 500 |
| 30% IBOA | | | | | | |
| Oligomer 2-4, | 3 | 7 | 47 | 44 A | 100 | 200 |
| 50% TRPGDA | | | | | | |

*Determination error of M_{W}, Mₙ and of MWD and η is 15%.
**η of an uncured formulation with diluent.

The high functionality of the urethane acrylates produced in accordance with the present invention affords the potential for providing cured materials that are highly crosslinked and tough; that is due to the fact that these products cannot build molecular weight through chain extension and, indeed, contain exactly one urethane linkage per acrylate group. Oligomers made with standard diisocyanate compounds, on the other hand (which of course have two urethane linkages per acrylate group, depending on the amount of chain extension) rapidly develop very high viscosities, and eventually results in gelled products, making them unsuitable, or indeed unusable. The UAO's produced by reacting IA and IMA with polyols of highf_{oH} demonstrate combinations of good physical properties and low viscosities that are unique to the present invention.

3. Comparative Properties of Standard UAOs Prepared from Polyol (f_{OH}=2), and of the Same Polyol Capped by IMA

Standard UAOs are prepared, as described above, using the following combinations of the polyether polypropylene glycol product Voranol 220-028, the aliphatic diisocyanate DesW, and the capping agents IPDI or HEMA: Voranol, IPDI, HEMA (designated Oligomer 3-1); Voranol, DesW, HEMA (designated Oligomer 3-2); and Voranol, IMA (designated Oligomer 3-3). Properties of the oligomers are summarized in the Table 7:

TABLE 7
Properties of oligomers prepared with Voranol*

| | Viscosity η, P | | | |
|---|---|---|---|---|
| | @ 25° C. | M_{w}, g/mol | Mₙ, g/mol | MWD |
| Oligomer 3-1 | 95 | 8,900 | 3600 | 2.47 |
| Oligomer 3-2 | 355 | 14,480 | 3,380 | 4.28 |
| Oligomer 3-3 | 16 | 6,400 | 4,130 | 1.55 |

*Determination error of the presented values is 15%.

The oligomers identified Table 7 were cured neat, in the manner described, to produced cured oligomers having the properties set forth in Table 8:

TABLE 8
Properties of cured oligomers prepared with Voranol*

| | Oligomer 3-1 | Oligomer 3-2 | Oligomer 3-3 |
|---|---|---|---|
| Tensile Strength, MPa | 0.9 | 1.7 | 0.11 |
| Elongation to break, % | 133 | 262 | 52 |
| Tensile Modulus, MPa | 0.8 | 0.65 | 2.75 |
| Hardness | 38 A | 33 A | 33 A |
| MEK double rubs | 10 | 6 | 12 |

*Determination error of the presented values is 15%.

The data in Table 8 show that the Oligomer 3-3 oligomer, made with IMA in one stage, has properties comparable to those of standard UAOs made with DIs. It is noted however that the Oligomer 3-3 product is much tackier, and has much better adhesion to glass, than either the Oligomer 3-2 or the Oligomer 3-1 products.
4. Comparative Properties of UAOs Prepared from Polyol (f_{OH}=2), and of the Same Polyol Capped by IA

In a first part of this example, the polyol Terathane 2000 was capped, in a one-stage reaction, with IA whereas, in a second part of the example, the reverse addition scheme described above was used to produce a UAO based on IPDI, HEA and the same polyol. More particularly IPDI was first reacted with HEA at 45° C., after which a stoichiometric amount of the polyol was added, and further reaction was effected 65° C. Two UAOs were prepared: Terathane, Iowa (designated Oligomer 4-1) and HEA, IPDI, Terathane, equivalents 1:2:1, respectively (designated Oligomer 4-2); the viscosity of Oligomer 4-2 was found to be much higher than that of Oligomer 4-1.

The foregoing oligomers were diluted and cured, in the manner described above, to produce products having the properties reported in Table 9:

TABLE 9
Properties of cured oligomer products prepared with Terathane 2000*

| | Oligomer | Oligomer | Oligomer | Oligomer |
|---|---|---|---|---|
| | 4-1, | 4-1, 50% | 4-2, | 4-2, 50% |
| | 30% IBOA | TRPGDA | 30% IBOA | TRPGDA |
| Tensile Strength, | 1.7 | 7.5 | 3 | 11 |
| MPa | | | | |
| Elongation at | 64 | 24 | 165 | 24.5 |
| break, % | | | | |
| Tensile Modulus, | 3.5 | 43 | 2 | 81 |
| MPa | | | | |
| MEK double | 9 | 47 | 60 | 80 |
| rubs | | | | |
| Hardness | 61 A | 87 A | 57 A | 42 D |
| Viscosity η**, P | 11 | 47.5 | 55 | 12 |

*Determination error of the presented values is 15%.
**η of an uncured formulation with diluent.

### 5. Effect of Molecular Weight on the Properties of Oligomers Capped by IMA

Molecular weight of polyol (f_{OH}=2) affects the final cured properties of standard UAOs, It was found in this invention that molecular weight of the starting polyol is much more effective in the final film properties. Standard UAOs with using DesW and HEMA, and IMA-capped oligomers were prepared in the manner hereinabove described, using the polyols PolyTHF 250 (MW˜250 g/mol), PolyTHF 650 (MW˜650 g/mol), PolyTHF 1000 (MW˜1000 g/mol), PolyTHF 2000 (MW˜2000 g/mol), and PolyTHF 2900 (MW˜29000 g/mol). PolyTHF 250 diol capped by IMA (designated as Oligomer 5-1A); PolyTHF 250 reacted with DesW and capped by HEMA (designated as Oligomer 5-1B).

It follows from the data in Table 10 that standard UAOs are much more viscous than their IA-capped analogues. The general observation can be made that IA- and IMA-capped oligomers have been found to exhibit much lower viscosities than standard UAOs of similar structure. That property is believed to be attributable, firstly, to the fact that IA/IMA-capped UAOs have only one polyol molecule in their structure, and thus lower molecular weights than standard UAOs; and secondly, to the fact that IA/IMA-capped UAOs have one-half the number of urethane (carbamate) links that are present in standard UAOs, which links form hydrogen bonds between standard UAO molecules, leading in turn to increased viscosities.

Oligomers were diluted and cured, as described, to produce products having the properties presented in Table 10 (wherein molecular weights are expressed a g/mol):

TABLE 10
Properties of cured formulations of oligomers 5-1 thru 5-5*

| | | | | Tensile | Elongation | Tensile |
|---|---|---|---|---|---|---|
| | Polyol | Isocyanate/ | Viscosity | Strength, | to break, | Modulus |
| | Mw | Methacrylate | η**, P | MPa | % | MPa |
| Oligomer 5-1A, | 250 | IMA | 2.3 | 41.6 | 5 | 1092 |
| 30% IBOA | | | | | | |
| Oligomer 5-1B, | 250 | DesW/HEMA | 1,030 | 56.4 | 6 | 1334 |
| 30% IBOA | | | | | | |
| Oligomer 5-2A, | 650 | IMA | 3 | 5.8 | 70 | 7.6 |
| 30% IBOA | | | | | | |
| Oligomer 5-2B, | 650 | DesW/HEMA | 150 | 32.3 | 98 | 35.2 |
| 30% IBOA | | | | | | |
| Oligomer 5-3A, | 1000 | IMA | 4.3 | 2.0 | 38 | 6.3 |
| 30% IBOA | | | | | | |
| Oligomer 5-3B, | 1000 | DesW/HEMA | 235 | 35.3 | 245 | 9.4 |
| 30% IBOA | | | | | | |
| Oligomer 5-4A, | 2000 | IMA | 10 | 0.5 | 18 | 3.9 |
| 30% IBOA | | | | | | |
| Oligomer 5-4B, | 2000 | DesW/HEMA | 500 | 16.8 | 353 | 2.4 |
| 30% IBOA | | | | | | |
| Oligomer 5-5A, | 2900 | IMA | 24 | 1.8 | 176 | 0.2 |
| 30% IBOA | | | | | | |
| Oligomer 5-5B, | 2900 | DesW/HEMA | 4150 | 17.2 | 441 | 2.2 |
| 30% IBOA | | | | | | |

*Determination error of the presented values is 15%.
**η of an uncured formulation with diluent.

It was found in this invention that as the molecular weight of the starting polyol was increased above 650 g/mol, IMA (and IA) capped oligomers result in a drastic drop in tensile strength as depicted in FIG. 1, which shows the optimal molecular weight range to be 250 to 650 g/mol.

### 6. Liquid Urethane (Meth)Acrylate Monomers

The invention further enables the capping, with monoisocynate-(meth)acrylate monomers, of hydroxyl-functional monomers to obtain liquid urethane products that would normally produce solid products with conventional isocyanate methods. We have found several applications for such novel liquid monomers as described below:

6.1. Water-Reducible Urethane Acrylate (UA) Synthesized with IA

Synthesis of water-miscible (also know as water-reducible) oligomers, or UV-curable polyurethane dispersions (UV-PUDs), was achieved by effecting reaction of low molecular weight diols having carboxylic functionality, such as 2,2-dihydroxymethyl butanoic acid (DMBA), Dimethylol acetic acid (DMAA), Dimethylol propionic acid (DMPA), Dimethylol pentanoic acid (dimethylol valeric acid) (DMVA), and Dimethylol hexanoic acid (dimethylol caproic acid) (DMCA), with IA. The absence of chain extension in such IA-capped, low molecular weight diols allows synthesis of water-soluble monomers. A unique advantage of IA capping is that monomers or oligomers so prepared contain at least one carboxylic group, which affords water solubility. High concentrations of carboxylic groups prevent precipitation of the monomer and, contrary to the usual requirement for UV-PUDs, avoids the need for detergents (surfactants).

A monomer, designated Monomer 6-1, was prepared by a reaction of DMBA with IA and represents an extreme example of the foregoing approach. Triethylamine (TEA) was added to the reaction product form a water-soluble quaternary ammonium salt having free acid groups, the structural formula for which is:

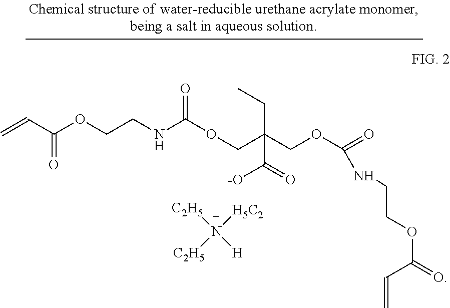

The resulting salt was found to be soluble in water in any concentration and, unlike other water-soluble oligomers, the Monomer 6-1 is colorless and transparent.

Upon admixture of 2.0 weight percent of Irgacure 819DW with an aqueous solution of Monomer 6-1, and evaporation of water, curing was effected. The cured product was found to be solvent-resistant, and to have a high tensile strength at break, as set forth in Table 11:

TABLE 11
Properties of aqueous solution and of cured water-reducible UA
Monomer 6-1

| | |
|---|---|
| Viscosity η, cP* | 25 |
| Tensile Strength, MPa** | 14 |
| Elongation at break, %** | 13 |
| Tensile Modulus, MPa** | 250 |
| MEK double rubs** | 200 |

*Determination error 10%.
**Determination error 15%.

Useful properties of conventional UV-PUDs are the result of achieving high oligomer molecular weights in aqueous solution. In contrast, the good physical properties of the cured Monomer 6-1 monomer are believed to be attributable to high crosslink density, due in turn to high concentrations of acrylate groups.

### 6.2. UA Monomer—Adhesion Promoter

Reaction of an excess of DMBA (1.5 moles) with IA (1.0 mole) leads to a mixture (designated Monomer 6-2) of two urethane acrylate monomers having the following chemical structures, which monomers are present in a molor ratio of approximately 1:1:

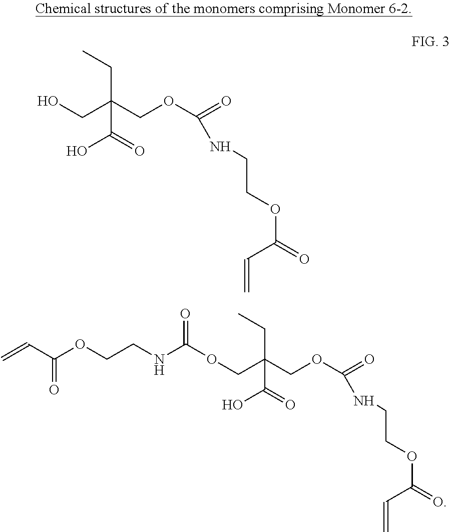

The Monomer 6-2 product is viscous, but pourable, at room temperature; it has a viscosity of 110 P at 50° C. The product is found to be a valuable additive to UAOs. For example, the addition 15% Monomer 6-2 to UAOs is found to lead to a substantial improvement in the adhesion to the cured mixture to stainless steel, as measured by the pick up test referred to hereinabove (Monomer 6-2 has free carboxyl and hydroxyl groups, which usually enhance adhesion of coatings to metals and other substrates). The addition of Monomer 6-2 is also found to increase tensile modulus, tensile strength, and chemical resistance of UAOs. Table 13 demonstrate the effects of Monomer 6-2 on the properties of formulations comprised of the UAO BR-582, referred to above, and a reactive diluent:

TABLE 13
Properties of liquid and cured formulations based on BR-582*

| | 55% BR-582, | | 35% BR-582, | |
|---|---|---|---|---|
| | 15% Monomer | BR-582, | 15% Monomer | BR-582, |
| | 6-2, | 30% | 6-2, 50% | 50% |
| | 30% IBOA | IBOA | TRPGDA | TRPGDA |
| Tensile | 24 | 21 | 19 | 13 |
| Strength, | | | | |
| MPa | | | | |
| Elongation at | 156 | 214 | 30 | 9 |
| break, % | | | | |
| Modulus, MPa | 94 | 37 | 805 | 325 |
| MEK double | >200 | 39 | 90 | 83 |
| rubs | | | | |
| Viscosity η, | 1,050 | 1,000 | 77.5 | 66 |
| P** | | | | |
| Hardness | 48 D | 61 D | 63 D | 67 D |

*Determination error of the presented values is 15%.
**η of an uncured formulation with diluent.

### 6.3. Copolymerizable Liquid Photoinitiators

Photoinitiators with hydroxyalkyl group (e.g., Irgacure 184 and Darocur 1173) can easily be reacted with IA or IMA to produce a copolymerizable photoinitiator, in accordance with the following reactions:

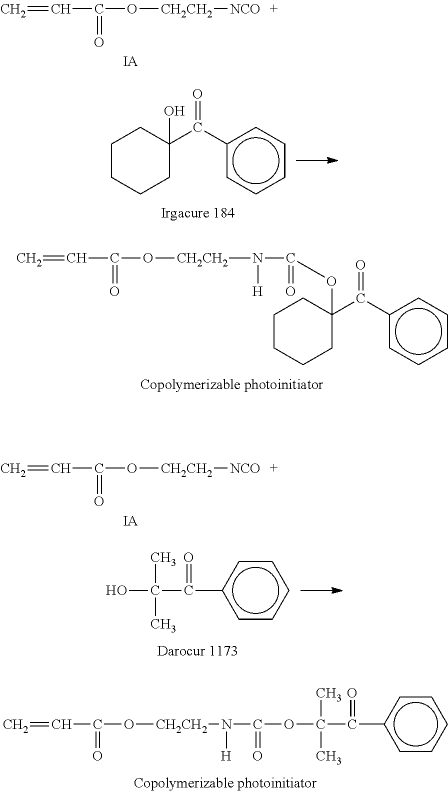

Copolymerizable photoinitiators have the known advantage of not leaching from the cured films in which they are contained (see Dietliker, J. A Compilation of Photoinitiators Commercially Available for UV Today; SITA: Edinburg 2002).

Adducts of Irgacure 184-IA, Irgacure 184-IMA, and Darocur 1173-IA were prepared, and used to effect curing in an acrylate formulation; the photoinitiators adducts were in the form of viscous liquids, at room temperature (which will generally be preferred to photoinitiators in solid form). Taking the Irgacure 184-IMA adduct as exemplary, it was found to be an efficient photoinitiator for effecting polymerization of many acrylates (while comparable, methacrylate formulations polymerize more slowly than the acrylates). The IMA-capped photoinitiators (as well as the IA-capped photoinitiators) were found to become part of the developing polymer network; this occurs however at later stages with the IMA-capped products than with the IA-capped products, thus making the IMA-capped initiators more efficient.

Thus, it can be seen that the present invention provides improved methods for synthesizing and utilizing monoisocyanate-(meth)acrylates, which enable expanded applications for such monomers; it provides novel isocyanate-based oligomers that lead to desirable properties in cured products in which they are employed; and it provides novel formulations and products containing such oligomers. More specifically, the invention provides isocyanate-based oligomers that are well suited for use in formulations that are curable to films, coatings, adhesives, and like solid products; that are of reduced viscosity in comparison to similar isocyanate-based oligomers; that afford significantly enhanced adhesion to certain substrates; and that may impart other desirable properties to products produced from formulations in which they are incorporated.

Having thus described the invention, what is claimed is:

1. A method for the production of useful urethane (meth)acrylate oligomers, without substantial gelation, comprising the steps:
forming a reaction mixture comprised of a monoisocyanate-(meth)acrylate monomer and a polyol having an hydroxyl functionality of at least 4, the amount of said polyol being not significantly in excess of the amount of said monoisocyanate(meth)acrylate monomer, on a stoichiometric basis; and
effecting reaction between said monoisocyanate-(meth)acrylate monomer and said polyol to produce a urethane (meth)acrylate oligomer that is substantially free of gelation and in which, on an equivalent basis, at least about 70 percent of the hydroxyl groups of said polyol are capped with said monoisocyanate-(meth)acrylate monomer.

2. The method of claim 1 wherein the amount of said polyol does not exceed the amount of said monoisocyanate-(meth)acrylate monomer by more than about 30 percent, on an hydroxyl equivalent basis.

3. The method of claim 1 wherein the amounts of said polyol and monoisocyanate-(meth)acrylate monomer are substantially stoichiometrically equivalent.

4. The method of claim 1 wherein said polyol is a dendrimer and said (meth)acrylate oligomer is a hyperbranched (meth)acrylate oligomer.

5. The method of claim 1 wherein said reaction is effected as a one-stage reaction.

6. The method of claim 1 wherein said monoiscocyanate-(meth)acrylate monomer is selected from the group consisting of 2-isocyanatoethyl acrylate and 2-isocyanatoethyl methacrylate.

7. A method for the production of a liquid derivative from a solid starting chemical, the physical states of said derivative and said starting chemical being determined at room temperature, comprising the steps:
forming a reaction mixture comprised of an monoisocyanate-(meth)acrylate monomer and a solid starting chemical that contains hydroxyl functionality; and
effecting reaction between said monoisocyanate-(meth)acrylate monomer and said solid starting chemical to produce a liquid derivative.

8. The method of claim 7 wherein said solid starting chemical is a low molecular weight diol.

9. The method of claim 8 wherein said solid starting chemical is selected from the group consisting of 2,2-dihydroxymethyl butanoic acid, dimethylol acetic acid, dimethylol propionic acid, dimethylol pentanoic acid, and dimethylol hexanoic acid.

10. The method of claim 7 wherein said monoiscocyanate-(meth)acrylate monomer is selected from the group consisting of 2-isocyanatoethyl acrylate and 2-isocyanatoethyl methacrylate.

11. The method of claim 9 wherein said starting chemical is 2,2-dihydroxymethyl butanoic acid and said monoisoyanate-(meth)acrylate monomer is 2-isocyanatoethyl acrylate, said acid being present in a molar ratio to said monomer of 1.5:1.0.

12. The method of claim 11 wherein said derivative produced is further reacted with triethylamine to produce a water-soluble quaternary ammonium salt.

13. The method of claim 7 wherein said solid starting chemical is a photoinitiator containing hydroxyalkyl groups.

14. A method for the production of useful, relatively low viscosity (meth)acrylate oligomers comprising the steps:
forming a reaction mixture comprised about 30 to 75 percent of a monoisocyanate-(meth)acrylate monomer, and about 25 to 70 percent of a polyol containing two or three hydroxyl groups, or a mixture of such polyols, said polyol having a molecular weight in the range 250 to 650 g/mol;
effecting reaction between said monoisocyanate-(meth)acrylate monomer and said polyol to produce a urethane (meth)acrylate oligomer having a viscosity not higher than about 150 MPa.

15. The method of claim 14 wherein said polyol is monomeric.

16. The method of claim 14 wherein said monoiscocyanate-(meth)acrylate monomer is selected from the group consisting of 2-isocyanatoethyl acrylate and 2-isocyanatoethyl methacrylate.

17. A urethane (meth)acrylate oligomer product produced by the method of claim 1.

18. A urethane (meth)acrylate-oligomer product produced by the method of claim 7.

19. A urethane (meth)acrylate oligomer product produced by the method of claim 14.

20. A formulation that is reactive to produce a solid polymeric product comprising a urethane (meth)acrylate oligomer product produced by the method of claim 1 and a polymerizable diluent reactive with said oligomer.

21. The formulation of claim 20 wherein said polymerizable diluent comprises a (meth)acrylate monomer.

22. The formulation of claim 20 containing about 70 to 50 weight percent of said urethane (meth)acrylate oligomer product and, conversely, about 30 to 50 weight percent of said reactive diluent.

23. The formulation of claim 20 additionally including a catalyst for inducing free radical polymerization.

24. The formulation of claim 23 wherein said catalyst is a photoinitiator or a thermal initiator

25. The formulation of claim 20 wherein said monoisocyanate-(meth)acrylate monomer employed in producing said urethane (meth)acrylate oligomer product is selected from the group consisting of 2-isocyanatoethyl acrylate and 2-isocyanatoethyl methacrylate.

26. A formulation that is reactive to produce a solid polymeric product comprising a urethane (meth)acrylate oligomer product produced by the method of claim 7 and a polymerizable diluent reactive with said oligomer.

27. The formulation of claim 26 wherein said polymerizable diluent comprises a (meth)acrylate monomer.

28. The formulation of claim 26 containing about 70 to 50 weight percent of said urethane (meth)acrylate oligomer product and, conversely, about 30 to 50 weight percent of said reactive diluent.

29. The formulation of claim 26 additionally including a catalyst for inducing free radical polymerization.

30. The formulation of claim 28 wherein said catalyst is a photoinitiator, or a thermal initiator.

31. A water-reducible urethane acrylate monomer having the chemical structure:

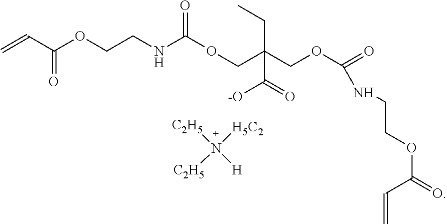

32. A mixture of two urethane acrylate monomers having the chemical structures:

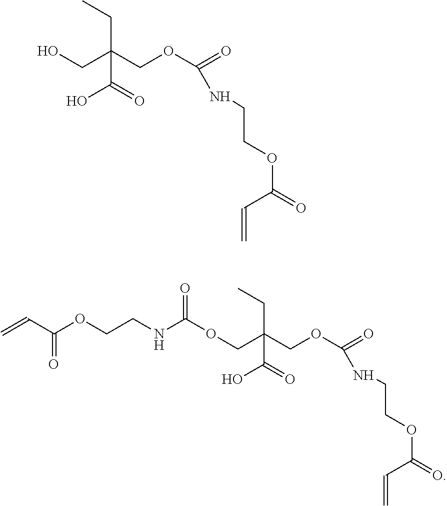

33. The mixture of claim 32 wherein said monomers are present in a substantially equimolar ratio.
